(19) United States
(12) Patent Application Publication
(10) Pub. No.: US 2003/0120230 A1
(43) Pub. Date: Jun. 26, 2003

(54) Absorbent article exhibiting improved fluid distribution

(76) Inventors: Andrea Susan Wulz, Roswell, GA (US); Melpo Lambidonis, Cumming, GA (US); Susan Carol Paul, Alpharetta, GA (US); Rebecca Willey Griffin, Woodstock, GA (US); Roland Columbus Smith JR., Gainesville, GA (US); David Craige Strack, Canton, GA (US); Eugenio Go Varona, Marietta, GA (US); Marlene Rebecca Waugaman, Alpharetta, GA (US); Tamara Lee Mace, Dorville, GA (US)

Correspondence Address: KIMBERLY-CLARK WORLDWIDE, INC., 401 NORTH LAKE STREET, NEENAH, WI, 54956

(21) Appl. No.: 10/026,862

(22) Filed: Dec. 20, 2001

(51) Int. Cl. A61F 13/15

(52) U.S. Cl. 604/368; 604/378

(57) ABSTRACT

The present invention relates to an absorbent article for absorbing fluids and exudates, such as urine. More particularly, the present invention relates to absorbent garments which are configured to absorb body exudates while also helping to reduce the relative humidity in the environment of the article, to reduce skin-exudate contact and to reduced undesired skin hydration.

The present invention relates to an absorbent article having a front waist section, a rear waist section, and an intermediate section which interconnects said front and rear waist sections. One embodiment of the invention generally includes: a topsheet; a backsheet; an absorbent material containing, at least in part, a superabsorbent; and a second material. One or more regions of the article contains less absorbent material than other regions of the article, and the second material is positioned in at least one of the one or more regions of article which contain less absorbent material.

### BACKGROUND OF THE INVENTION

Many known diaper configurations employ absorbent materials located between a liquid pervious topsheet and a vapor and liquid impermeable backsheet. Such backsheets are well suited to prevent the migration of liquid waste from the absorbent materials to the outer garments of a wearer. Unfortunately, the use of liquid and vapor impermeable backsheets can result in a high degree of humidity within the diaper when in use which may result in relatively high skin hydration levels. The occlusive, moist environment inside diapers incorporating such backsheets can promote the viability of microorganisms, including Candida albicans, which can undesirably lead to the onset of diaper dermatitis (diaper rash).

Diaper dermatitis can afflict almost every infant at some time during the diaper wearing years. The most severe form of this condition is usually caused by secondary infection with the fungi Candida albicans. Although other factors influence the pathogenesis of this fungi, one critical factor is the relative humidity within the diaper which is directly related to the occlusion or semi-occlusion of the diaper area.

In order to reduce the humidity level within diapers, breathable polymer films have been employed as outer covers for absorbent garments, such as disposable diapers. The breathable films are typically constructed with micropores to provide desired levels of liquid impermeability and air permeability. Other disposable diaper designs have been arranged to provide breathable regions in the form of breathable panels or perforated regions in otherwise vapor-impermeable backsheets to help ventilate the garment.

Conventional absorbent articles, such as those described above, have not been completely satisfactory. For example, articles which employ perforated films or breathable panels can exhibit excessive leakage of liquids from the article and can excessively soil the wearer's inner or outer garments in the regions of the perforations or panels. In addition, when the absorbent material of the article becomes loaded with liquid, the wet absorbent can block the escape of moisture from the wearer's skin. Such absorbent garment designs have not been able to maintain a high level of breathability when wet to sufficiently reduce the hydration of the wearer's skin. As a result, the wearer's skin has remained susceptible to rashes, abrasion and irritation.

While a number of improvements have been made to conventional absorbent articles, a frequent problem still encountered with these articles is the tendency for an insult fluid to pool or accumulate in one or more regions of the article. Sometimes the accumulation occurs on the surface of the articles and sometimes the accumulation is experienced on the inside of the article. In either instance the accumulation will generally occur at or in the insult or target area of the article. This accumulation at the target or insult area generally reduces the article's ability to readily accept additional insults, and thus may affect the article's leakage and dryness characteristics. While some absorbent articles having wicking materials included therein, the fluid which insults these articles still has a tendency to accumulate in the target or insult area of the product. Generally, speaking conventional absorbent articles would benefit (e.g. improvement in leakage and dryness) from better and faster fluid distribution such that the article, and more specifically, the target or insult area of the article, is more receptive to subsequent insults and such that the wearer is less likely to be exposed to the moisture in the article.

### SUMMARY OF THE INVENTION

The present invention relates to an absorbent article for absorbing fluids and exudates, such as urine. More particularly, the present invention relates to absorbent garments, such as disposable diapers and adult incontinence garments, which are configured to absorb body exudates while also helping to reduce the relative humidity in the environment the article, to reduce skin-exudate contact and to provide reduced skin hydration.

More specifically the present invention relates to an absorbent article having a front waist section, a rear waist section, and an intermediate section which interconnects the front and rear waist sections, the intermediate section including a crotch region. The absorbent article more particularly includes: (a) an absorbent material having, at least in part, a superabsorbent; and (b) a second material or insert; wherein one or more regions of the article contains less absorbent material than other regions of the article, and wherein the second material is positioned in at least one of the one or more regions of article which contain less absorbent material. The reduction of absorbent material and the positioning of the second material or insert in one or more regions of the article will promote the flow of an insulting fluid from the one or more regions of the article containing less absorbent material to other regions of the article so as to change the fluid profile in the article. The article is also designed such that the fluid will be moved out of the insult or target area of the article in less than about two minutes, which is significantly faster than traditional absorbent articles. In one embodiment of the present invention the absorbent will be absent from one or more regions of the article, and desirably absent from the crotch region of the article. The present invention may further include a surge material and/or a vapor barrier. The surge material is desirably positioned between the topsheet and a second, desirably superabsorbent-free, material or the absorbent material while the vapor barrier is desirably positioned between the absorbent and the topsheet (or surge, if present) so as to reduce the amount of evaporative moisture the skin of a wearer is exposed to.

Another embodiment of the present invention relates to a composite system defining a front waist section, a rear waist section, and an intermediate section which interconnects the front and rear waist sections, the intermediate section having or forming a crotch region. The composite system includes (a) a superabsorbent-free material or insert; and (b) an absorbent, containing, at least in part a superabsorbent, wherein the absorbent (b) is absent from one or more regions of the article. The insert or superabsorbent-free material is positioned in one or more regions of the article adjacent the absorbent. Desirably the absence of absorbent in one or more regions of the article and the presence of the superabsorbent-free material in those regions promotes a reduction in the time needed to move the insult fluid from the superabsorbent-free material to the absorbent. Desirably the insult will be removed from the superabsorbent-free material to the absorbent material in less than about 2 minutes.

Yet another embodiment of the present invention defines a disposable absorbent article having a vapor permeable backsheet with a Water Vapor Transmission Rate (WVTR) of at least about 1000 grams per square meter per 24 hours, a liquid permeable topsheet; an absorbent body located between the backsheet and the topsheet, the absorbent body being located in one or more regions of the article; and a second material located between the backsheet and the topsheet, the second material being positioned in one or more regions of the article where the absorbent body is not present. The second material allows fluid to be moved away from an insult area of the absorbent article to a region of the absorbent article where the absorbent is located so as to change the fluid profile in the diaper. The change in the fluid profile of the diaper may provide for a reduction in skin-exudate contact and may also provide reduced saturation in the insult or target area. Desirably, the absorbent body or material will be absent from the insult area of the absorbent article. The disposable article may also further include a vapor barrier positioned between the absorbent or surge and the topsheet, wherein the vapor barrier reduces or minimizes the moisture (including vapor) movement between from the absorbent core into environment between the absorbent article and the user's skin.

### BRIEF DESCRIPTION OF THE DRAWINGS

The invention will be more fully understood and further advantages will become apparent when reference is made to the following detailed description of the invention and the accompanying drawings, in which:

FIG. 1 representatively shows a partially cutaway, top plan view of an absorbent article according to one embodiment of the invention;

FIG. 2 representatively shows a sectional view of the absorbent article of FIG. 1 taken along line 2-2.

FIG. 3 representatively shows a partially cutaway, top plan view of an absorbent body for an absorbent article according to another embodiment of the invention;

FIG. 4 representatively shows a sectional view of the absorbent body of FIG. 3 taken along line 4-4.

FIG. 5 representatively illustrates the cradle used to the test the materials described in Examples 1-7.

### DEFINITIONS

As used herein the following terms have the specified meanings, unless the context demands a different meaning, or a different meaning is expressed; also, the singular generally includes the plural, and the plural generally includes the singular unless otherwise indicated.

As used herein, all percentages, ratios and proportions are by weight unless otherwise specified.

As used herein, the term “biconstituent fibers” refers to fibers which have been formed from at least two polymers extruded from the same extruder as a blend. The term “blend” is defined below. Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Biconstituent fibers are sometimes also referred to as multiconstituent fibers. Fibers of this general type are discussed in, for example, U.S. Pat. No. 5,108,827 to Gessner. Bicomponent and biconstituent fibers are also discussed in the textbook Polymer Blends and Composites by John A. Manson and Leslie H. Sperling, copyright 1976 by Plenum Press, a division of Plenum Publishing Corporation of New York, IBSN 0-306-30831-2, at pages 273 through 277.

As used herein the term “blend” means a mixture of two or more polymers while the term “alloy” means a sub-class of blends wherein the components are immiscible but have been compatibilized. “Miscibility” and “immiscibility” are defined as blends having negative and positive values, respectively, for the free energy of mixing. Further, “compatibilization” is defined as the process of modifying the interfacial properties of an immiscible polymer blend in order to make an alloy.

As used herein, the terms “comprises”, “comprising” and other derivatives from the root term “comprise” are intended to be inclusive or open-ended terms that specify the presence of any stated features, elements, integers, steps, or components, but do not preclude the presence or addition of one or more other features, elements, integers, steps, components, or groups thereof. Accordingly, the term “comprising” encompasses the more restrictive terms “consisting essentially of” and “consisting of.”

As used herein, the term “conjugate fibers” refers to fibers which have been formed from at least two polymers extruded from separate extruders but spun together to form one fiber. Conjugate fibers are also sometimes referred to as multicomponent or bicomponent fibers. The polymers are usually different from each other though conjugate fibers may be monocomponent fibers. The polymers are arranged in substantially constantly positioned distinct zones across the cross-section of the conjugate fibers and extend continuously along the length of the conjugate fibers. The configuration of such a conjugate fiber may be, for example, a sheath/core arrangement wherein one polymer is surrounded by another or may be a side by side arrangement, a pie arrangement or an “islands-in-the-sea” arrangement. Conjugate fibers are taught in U.S. Pat. No. 5,108,820 to Kaneko et al., U.S. Pat. No. 5,336,552 to Strack et al., and U.S. Pat. No. 5,382,400 to Pike et al. For two component fibers, the polymers may be present in ratios of 75/25, 50/50, 25/75 or any other desired ratios. The fibers may also have shapes such as those described in U.S. Pat. Nos. 5,277,976 to Hogle et al., and 5,069,970 and 5,057,368 to Largman et al., hereby incorporated by reference in their entirety, which describe fibers with unconventional shapes.

As used herein, the term “disposable” includes being disposed of after a single use and not intended to be washed and reused.

As used herein, the term “fabric” refers to all of the woven, knitted and nonwoven fibrous webs.

The terms “front” and “back” are used throughout this description to designate relationships relative to the garment itself, rather than to suggest any position the garment assumes when it is positioned on a wearer.

As used herein, the terms “inward” and “outward” refer to positions relative to the center of an absorbent garment, and particularly transversely and/or longitudinally closer to or away from the longitudinal and transverse center of the absorbent garment.

As used herein, the term “layer” when used in the singular can have the dual meaning of a single element or a plurality of elements.

As used herein, the term “liquid” means a non-particulate substance and/or material that flows and can assume the interior shape of a container into which it is poured or placed.

As used herein, the term “liquid communication” means that liquid is able to travel from one layer to another layer, or one location to another within a layer.

As used herein, the terms “longitudinal” and “transverse” have their customary meanings. The longitudinal axis lies in the plane of the article when laid flat and fully extended and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

As used herein the term “microfibers” means small diameter fibers having an average diameter not greater than about 75 microns, for example, having an average diameter of from about 0.5 microns to about 50 microns, or more particularly, microfibers may have an average diameter of from about 2 microns to about 40 microns. Another frequently used expression of fiber diameter is denier, which is defined as grams per 9000 meters of a fiber and may be calculated as fiber diameter in microns squared, multiplied by the density in grams/cc, multiplied by 0.00707. A lower denier indicates a finer fiber and a higher denier indicates a thicker or heavier fiber. For example, the diameter of a polypropylene fiber given as 15 microns may be converted to denier by squaring, multiplying the result by 0.89 g/cc and multiplying by 0.00707. Thus, a 15 micron polypropylene fiber has a denier of about 1.42 (15²×0.89×0.00707=1.415). Outside the United States the unit of measurement is more commonly the “tex”, which is defined as the grams per kilometer of fiber. Tex may be calculated as denier/9.

As used herein “multilayer laminate” means a laminate having multiple layers. For example, some of the layers may be spunbond and some meltblown, such as a spunbond/meltblown/spunbond (SMS) laminate and others as disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. Such a laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such exemplary fabrics usually have a basis weight of from about 0.1 to 12 osy (6 to 400 gsm), or more particularly from about 0.75 to about 3 osy. Multilayer laminates may also have various numbers of meltblown layers or multiple spunbond layers in many different configurations and may include other materials like films (F) or coform materials, e.g. SMMS, SM, SFS, etc.

As used herein the terms “nonwoven” and “nonwoven fabric or web” mean a web having a structure of individual fibers, filaments or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein, the term “personal care product” or “personal care absorbent product” means diapers, training pants, swim wear, absorbent underpants, baby wipes, incontinence products and devices, sanitary wipes, wet wipes, feminine hygiene products, absorbent pads, mortuary pads, veterinary pads, wound dressings and bandages, and the like.

As used herein, the terms “region” or “area” refer generally a part of an object or material which can be larger or smaller than a section or portion thereof, and can be made up of one or more sections, portions or parts thereof. As used herein, the terms “region”, “area” are generally interchangeable.

As used herein, the term “skin” refers to the outermost exposed layer of a mammal's dermis or epidermis, and may be a wound.

As used herein the term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

As used herein, the term “target area” refers to the area or position on or in a personal care product where an insult is normally delivered by a wearer.

As used herein, a substantially fluid or liquid impermeable material is constructed to provide a hydrohead of at least about 60 centimeters (cm), desirably at least about 80 cm, and more desirably at least about 100 cm. A suitable technique for determining the hydrohead value is the Hydrostatic Pressure Test which is described in further detail herein below.

As used herein, a substantially vapor permeable material is constructed to provide a water vapor transmission rate (WVTR) of at least about 100 g/m²/24 hr, desirably at least about 250 g/m²/24 hr, and more desirably at least about 500 g/m²/24 hr. A suitable technique for determining the WVTR value is the Water Vapor Transmission Rate Test which is described in further detail herein below.

These terms may be defined with additional language in the remaining portions of the specification.

### Test Methods

Hydrostatic Pressure Test

The Hydrostatic Pressure Test is a measure of the liquid barrier properties of a material. In general, the Hydrostatic Pressure Test determines the height of water (in centimeters) in a column which the material will support before a predetermined amount of water passes through. A material with a higher hydrohead value indicates it is a greater barrier to liquid penetration than a material having a lower hydrohead value. The Hydrostatic Pressure Test is performed according to Method 5514-Federal Test Methods Standard No. 191A.

Water Vapor Transmission Test

A suitable technique for determining the WVTR (water vapor transmission rate) value of a material is as follows. For the purposes of the present invention, 3-inch diameter (76 millimeter) circular samples are cut from the test material and from a control material, Celguard® 2500 (Hoechst Celanese Corporation). Two or three samples are prepared for each material. Test cups used for testing are cast aluminum, flanged, 2 inches deep and come with a mechanical seal and neoprene gasket. The cups are distributed by Thwing-Albert Instrument Company, Philadelphia, Pa., under the designation Vapometer cup #681. One hundred milliliters of distilled water are poured into each Vapometer cup, and each of the individual samples of the test materials and control material are placed across the open top area of an individual cup. Screw-on flanges are tightened to form a seal along the edges of the cups leaving the associated test material or control material exposed to the ambient atmosphere over a 62 millimeter diameter circular area (an open, exposed area of about 30 cm²). The cups are then weighed, placed on a tray, and set in a forced air oven set at 100° F. (38° C.). The oven is a constant temperature oven with external air circulating through it to prevent water vapor accumulation inside. A suitable forced air oven is, for example, a Blue M Power-O-Matic 60 oven distributed by Blue M Electric Co. of Blue Island, Ill. After 24 hours, the cups are removed from the oven and weighed. The preliminary, test WVTR value is calculated as follows:

$$Test WVTR = [ \frac{( grams weight loss over 24 hours ) \times 7571}{24} ] ( g \text{/} {m}^{2} \text{/} 24 hours )$$

The relative humidity within the oven is not specifically controlled. Under predetermined set conditions of 100° F. and ambient relative humidity, the WVTR for Celguard 2500 has been determined to be 5000 g/m²124 hours. Accordingly, Celguard 2500 is run as a control sample with each test. Celguard 2500 is a 0.0025 cm thick film composed of a microporous polypropylene.

Bench Test Method

The articles and systems of the present invention were examined using a cradle. The cradle that is used for the testing is acrylic and is formed to simulate the curvature of a user such as an infant. Such a cradle is illustrated in FIG. 5. The cradle has a width into the page of the drawing as shown of 33 cm and the ends are blocked off, a height of 19.5 cm, an inner distance between the upper arms of 28 cm and an angle between the upper arms of 60 degrees. The cradle has a 6.5 mm wide slot at the lowest point running the length of the cradle into the page. The material to be tested is placed in the cradle with the topsheet up and the backsheet towards the acrylic cradle. The target or crotch is centered over the slot at the lowest point of the cradle. The ends of the material are placed above the crotch, to simulate the position of the product on a user. A first insult, 60 ml Blood Bank Saline (pH 7.0-7.2, 8.5 g/L of A.C.S. grade Sodium Chloride, CAS #7647-14-5 in ultrapure reagent grade water, CAS #7732-18-5), is introduced into the product using a Masterflex Digi-Staltic® 7526-00 pump with Masterflex 6409-17 tubing (both available from Cole-Parmer Instrument Company, Vernon Hills, Ill.) connected to a spray nozzle with a 3 mm orifice. The insult is introduced at a rate of 15 ml/sec. One minute after the insult, the material is removed from the cradle and placed flat on an x-ray unit. An x-ray is taken of the xy-plane of the diaper and is analyzed for fluid distribution. The material is placed back in the cradle in substantially the same position as the original placement. A second insult of 60 ml of saline is introduced into the diaper after 20 minutes in the cradle. One more minute after the second insult is complete, the product is removed and x-rayed a second time and analyzed for fluid distribution. The x-ray system was operated with an exposure time of 5 seconds, with a tube voltage of 30 Kv and current of 12 mA. Prior to each x-ray, a metal rod was placed across the entire width or cross-direction of the product at the point of insult such that the insult point was discernable in the x-ray picture.

The x-ray systems used in these Examples are available from Tronix Inc. of 31 Business Park Drive, Branford, Conn. 06045 as Model No. 10561 HF 100 with an appropriate enclosure. This system uses BIO-SCAN OPTIMATE version 6.2 software, available from Optumus, Inc., of Ft. Collins, Colo.

### DETAILED DESCRIPTION OF THE INVENTION

The following detailed description will be made in the context of a disposable diaper article which is adapted to be worn by infants about the lower torso. It is readily apparent, however, that the absorbent article and composite system of the present invention would also be suitable for use as other types of absorbent articles, training pants, swim wear, absorbent underpants, incontinence products and devices, feminine hygiene products, absorbent pads, hygiene products and the like. In addition, the invention will be described in the context of its various configurations. It should be appreciated that alternative arrangements of the invention can comprise any combination of such configurations. As such, the use of a desired embodiment, a diaper, for ease in understanding and describing the invention is not intended to, in any manner, limit the scope of the invention.

The absorbent articles and composites systems of present invention advantageously exhibit improved fluid distribution, a decrease in fluid movement time (as described in more detail herein), and reduced skin-exudate contact when compared to conventional absorbent articles. Thus, wearers of absorbent articles of the different aspects of the present invention may experience reduced skin hydration which renders the skin less susceptible to the viability of microorganisms which can lead to a reduction in the incidence of skin irritation and rash. It has been discovered that the ability of the absorbent articles of the present invention to exhibit a low incidence of skin irritation and rash on the wearer's skin during use depends, at least in part, on the amount of fluid in contact with the skin of the wearer. Moreover, it has been further discovered that the achievement of such low levels of skin irritation and rash further depends on the ability of the product to maintain a low relative humidity in the environment of the product. The relative humidity of the environment of the product is intended to include that which is not only in the product itself, but also that area between the user and the article or product.

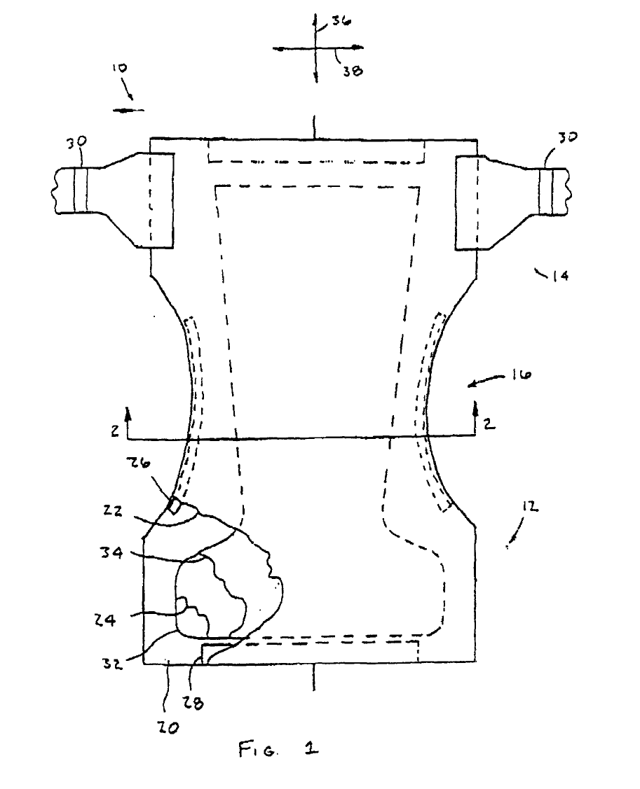

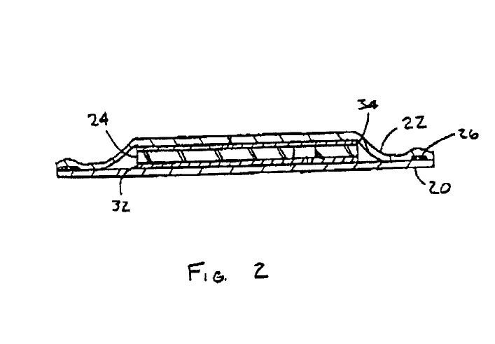

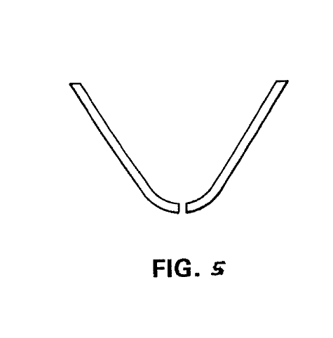

Examples of suitable constructions of absorbent articles for use in the present invention are described below and representatively illustrated in FIGS. 1-4. FIG. 1 is a representative plan view of an integral absorbent garment article, such as disposable diaper 10, of the present invention in its flat-out, uncontracted state (i.e., with all elastic induced gathering and contraction removed). Portions of the structure are partially cut away to more clearly show the interior construction of diaper 10, and the surface of the diaper which contacts the wearer is facing the viewer. FIG. 2 representatively shows a sectional view of the absorbent article of FIG. 1 taken along line 2-2. With reference to FIGS. 1 and 2, the disposable diaper 10 generally defines a front waist section 12, a rear waist section 14, and an intermediate section 16 which interconnects the front and rear waist sections. The front and rear waist sections include the general portions of the article which are constructed to extend substantially over the wearer's front and rear abdominal regions, respectively, during use. The intermediate section of the article includes the general portion of the article which is constructed to extend through the wearer's crotch region between the legs.

The absorbent article includes a vapor permeable backsheet 20, a liquid permeable topsheet 22, and an absorbent body or material 24, such as an absorbent pad or the like, which is located between the backsheet 20 and the topsheet 22. The backsheet 20 defines a length and a width which, in the illustrated embodiment, coincide with the length and width of the diaper 10. The absorbent body 24 generally defines a length and width which are less than the length and width of the backsheet 20, respectively. Thus, marginal portions of the diaper 10, such as marginal sections of the backsheet 20, may extend past the terminal edges of the absorbent body 24. It is also contemplated that the absorbent body 24 may be made up of one or more sections of absorbent material and which may or may not be adjacent or in fluid communication with the other sections. In the illustrated embodiments, for example, the backsheet 20 extends outwardly beyond the terminal marginal edges of the absorbent body 24 to form side margins and end margins of the diaper 10. The topsheet 22 is generally coextensive with the backsheet 20 but may optionally cover an area which is larger or smaller than the area of the backsheet 20, as desired. The backsheet 20 and topsheet 22 are intended to face the garment and body of the wearer, respectively, while in use.

The permeability of the backsheet is selected to enhance the breathability of the absorbent article to reduce the hydration of the wearer's skin during use without allowing excessive condensation of vapor on the garment facing surface of the backsheet 20 which can undesirably dampen the wearer's clothes.

To provide improved fit and to help reduce leakage of body exudates from the diaper 10, the diaper side margins and end margins may be elasticized with suitable elastic members, such as single or multiple strands of elastic. The elastic strands may be composed of natural or synthetic rubber and may optionally be heat shrinkable or heat elasticizable. For example, as representatively illustrated in FIGS. 1 and 2, the diaper 10 may include leg elastics 26 which are constructed to operably gather and shirr the side margins of the diaper 10 to provide elasticized leg bands which can closely fit around the legs of the wearer to reduce leakage and provide improved comfort and appearance. Similarly, waist elastics 28 can be employed to elasticize the end margins of the diaper 10 to provide elasticized waists. The waist elastics are configured to operably gather and shirr the waist sections to provide a resilient, comfortably close fit around the waist of the wearer. In the illustrated embodiments, the elastic members are illustrated in their uncontracted, stretched condition for the purpose of clarity.

Fastening means, such as hook and loop fasteners 30, are employed to secure the diaper on a wearer. Alternatively, other fastening means, such as buttons, pins, snaps, adhesive tape fasteners, cohesives, mushroom-and-loop fasteners, or the like, may be employed.

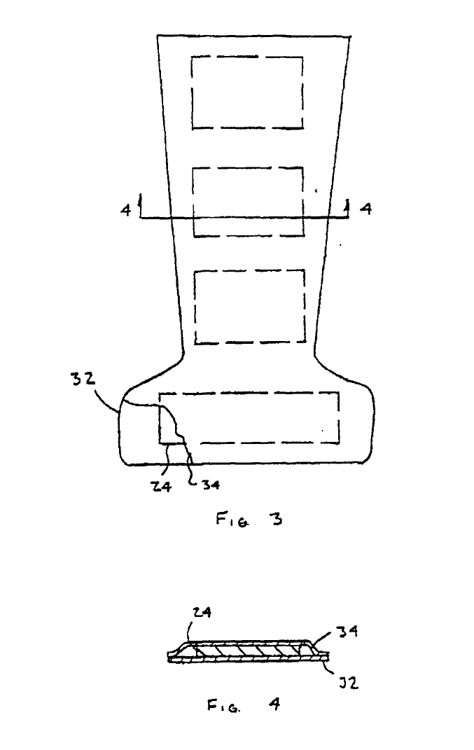

As indicated above, and as illustrated in FIG. 3, a diaper of the present invention may have one or more regions thereof which contain less absorbent material than other regions of the article. In one or more embodiments, the absorbent may be absent from one or more regions of the article, as shown in FIG. 3. Desirably, the absorbent will be absent from the insult or target area of the article, which is desirably the crotch region of the article.

The absorbent article may also include a second material or insert 25. While the second material may selected from a wide variety of materials, the material is desirably superabsorbent-free, although some embodiments of the present invention are contemplated to include some superabsorbent particles in the second material. Suitable materials which can be used as or in the second material, include, but are not limited to tissue, airlaid or surge materials, such as those used in surge layer 34 or the like.

The diaper 10 may further include other layers between the absorbent body 24 and the topsheet 22 or backsheet 20. For example, as representatively illustrated in FIGS. 1 and 2, the diaper 10 may include a ventilation or spacer layer 32 located between the absorbent body 24 and the backsheet 20 to insulate the backsheet 20 from the absorbent body 24 to improve air circulation and effectively reduce the dampness of the garment facing surface of the backsheet 20. The ventilation layer 32 may also assist in distributing fluid exudates to portions of the absorbent body 24 which do not directly receive the insult. The diaper 10 may also include a surge material or surge management layer 34 located between the topsheet 22 and the absorbent body 24 to prevent pooling of the fluid exudates and further improve air exchange and distribution of the fluid exudates within the diaper 10. In one or more embodiments, it may be desirable for the surge management layer 34 to be superabsorbent-free or substantially superabsorbent-free. In alternative embodiments, it may be necessary for the surge to have an increased capacity which may be achieved by the inclusion or incorporation of superabsorbent particles or the like. The present invention may further include a vapor barrier. The vapor barrier is desirably positioned between the absorbent and the topsheet (or surge, if present) so as to reduce the amount of evaporative moisture the skin of a wearer is exposed to. The vapor barrier may be a film, nonwoven, nonwoven/film laminate, foam or the like. Desirably the vapor barrier is, at least in part, a breathable film, but may be any other suitable breathable barrier known to those skilled in the art. Furthermore, it is contemplated that the vapor barrier may be apertured. It is also contemplated that the vapor barrier may be zoned or selectively applied or positioned in one or more regions of the article.

The diaper 10 may be of various suitable shapes. For example, the diaper may have an overall rectangular shape, T-shape or an approximately hour-glass shape. In the shown embodiment, the diaper 10 has a generally I-shape. The diaper 10 further defines a longitudinal direction 36 and a lateral direction 38. Other suitable diaper components which may be incorporated in absorbent articles of the present invention include containment flaps, waist flaps, elastomeric side panels, and the like which are generally known to those skilled in the art.

Examples of diaper configurations suitable for use in connection with the instant application which may include other diaper components suitable for use on diapers are described in U.S. Pat. No. 4,798,603 issued Jan. 17, 1989, to Meyer et al.; U.S. Pat. No. 5,176,668 issued Jan. 5, 1993, to Bernardin; U.S. Pat. No. 5,176,672 issued Jan. 5, 1993, to Bruemmer et al.; U.S. Pat. No. 5,192,606 issued Mar. 9, 1993, to Proxmire et al., and U.S. Pat. No. 5,509,915 issued Apr. 23, 1996 to Hanson et al., the disclosures of which are herein incorporated by reference in their entirety.

The various components of the diaper 10 are integrally assembled together employing various types of suitable attachment means, such as adhesive, sonic bonds, thermal bonds or combinations thereof. In the shown embodiment, for example, the topsheet 22 and backsheet 20 are assembled to each other and to the absorbent body 24 with lines of adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, other diaper components, such as the elastic members 26 and 28, fastening members 30, and ventilation and surge layers 32 and 34 may be assembled into the diaper article by employing the above-identified attachment mechanisms.

The backsheet 20 of the diaper 10, as representatively illustrated in FIGS. 1 and 2, is typically composed of a substantially vapor permeable material. The backsheet 20 may be generally constructed to be permeable to at least water vapor and may have a water vapor transmission rate of at least about 1000 g/m²/24 hr, desirably at least about 1500 g/m²/24 hr, more desirably at least about 2000 g/m²/24 hr, and even more desirably at least about 2500 g/m²/24 hr. For example, the backsheet 20 may define a water vapor transmission rate of from about 1000 to about 5000 g/m²/24 hr. Materials which have a water vapor transmission rate less than those above usually do not allow a sufficient amount of air exchange and can undesirably result in increased levels of skin hydration if no other means of humidity reduction within the diaper is available.

In one or more embodiments the backsheet 20 is also desirably substantially liquid impermeable. For example, the backsheet may be constructed to provide a hydrohead value of at least about 60 cm, desirably at least about 80 cm, and more desirably at least about 100 cm when subjected to the Hydrostatic Pressure Test. Materials which have hydrohead values less than those above undesirably result in the strike through of liquids during use. Such fluid strike through can undesirably result in a damp, clammy feeling on the backsheet 20 during use.

The backsheet 20 may be composed of any suitable materials which either directly provide the above desired levels of liquid impermeability and air permeability or, in the alternative, materials which can be modified or treated in some manner to provide such levels. In one embodiment, the backsheet 20 may be a nonwoven fibrous web constructed to provide the required level of liquid impermeability. For example, a nonwoven web composed of spunbonded or meltblown polymer fibers may be selectively treated with a water repellent coating or laminated with a liquid impermeable, vapor permeable polymer film to provide the backsheet 20. In a particular embodiment of the invention, the backsheet 20 may comprise a nonwoven web composed of a plurality of randomly deposited hydrophobic thermoplastic meltblown fibers which are sufficiently bonded or otherwise connected to one another to provide a substantially vapor permeable and substantially liquid impermeable web. The backsheet 20 may also comprise a vapor permeable nonwoven layer which has been partially coated or otherwise configured to provide liquid impermeability in selected areas. Desirably, the backsheet will have a Water Vapor Transmission Rate of at least 2,500 g/m²/24 hr.

Examples of suitable materials for the backsheet 20 are also described in U.S. Pat. No. 5,482,765 issued Jan. 9, 1996 in the name of Bradley et al. and entitled “Nonwoven Fabric Laminate With Enhanced Barrier Properties”; U.S. Pat. No. 5,879,341 issued Mar. 9, 1999 in the name of Odorzynski et al. and entitled “Absorbent Article Having A Breathability Gradient”; U.S. Pat. No. 5,843,056 issued Dec. 1, 1998, in the name of Good et al. and entitled “Absorbent Article Having A Composite Breathable Backsheet”; and U.S. Pat. No. 6,309,736 issued Oct. 30, 2001 in the name of McCormack et al. and entitled “Low Gauge Films And Film/Nonwoven Laminates”, the disclosures of which are herein incorporated by reference in their entirety.

In a particular embodiment, the backsheet 20 is provided by a highly breathable laminate and more particularly by a microporous film/nonwoven laminate material comprising a spunbond nonwoven material laminated to a microporous film. The spunbond nonwoven comprises filaments of about 1.8 denier extruded from polypropylene and defines a basis weight of from about 17 to about 25 grams per square meter. The film comprises a cast coextruded film having calcium carbonate-filled linear low polyethylene microporous core and ethylene vinyl acetate and Catalloy™ polypropylene (Catalloy™ 357P), available from Basell (having offices in Wilmington, Del.), blended skin layer having a basis weight of about 58 grams per square meter prior to stretching. The film is preheated, stretched and annealed to form the micropores and then laminated to the spunbond nonwoven. The resulting microporous film/nonwoven laminate based material has a basis weight of from about 30 to about 60 grams per square meter and a water vapor transmission rate of from about 3000 to about 5000 g/m²/24 hr. Examples of such film/nonwoven laminate materials are described in more detail in U.S. Pat. No. 6,309,736 issued Oct. 30, 2001 in the name of McCormack et al. and entitled “Low Gauge Films And Film/Nonwoven Laminates”, the disclosure of which has been incorporated by reference in its entirety.

The topsheet 22, as representatively illustrated in FIGS. 1 and 2, suitably presents a bodyfacing surface which is compliant, soft feeling, and nonirritating to the wearer's skin. Further, the topsheet 22 may be less hydrophilic than the absorbent body 24, to present a relatively dry surface to the wearer, and may be sufficiently porous to be liquid permeable, permitting liquid to readily penetrate through its thickness. A suitable topsheet 22 may be manufactured from a wide selection of web materials, such as porous foams, reticulated foams, apertured plastic films, natural fibers (for example, wood or cotton fibers), synthetic fibers (for example, polyester or polypropylene fibers), or a combination of natural and synthetic fibers. The topsheet 22 is suitably employed to help isolate the wearer's skin from liquids held in the absorbent body 24.

Various woven and nonwoven fabrics can be used for the topsheet 22. For example, the topsheet may be composed of a meltblown or spunbonded web of polyolefin fibers. The topsheet may also be a bonded-carded web composed of natural and/or synthetic fibers. The topsheet may be composed of a substantially hydrophobic material, and the hydrophobic material may, optionally, be treated with a surfactant or otherwise processed to impart a desired level of wettability and hydrophilicity. In a particular embodiment of the present invention, the topsheet 22 comprises a nonwoven spunbond, polypropylene fabric composed of about 2.2-2.8 denier fibers formed into a web having a basis weight of about 17 grams per square meter and a density of about 0.11 gram per cubic centimeter. Such a topsheet 22 may be surface treated with an effective amount of a surfactant such as about 0.3 weight percent of a surfactant commercially available from Uniqema under the trade designation AHCOVEL BASE N-62.

In one embodiment, no surfactant will be added to or incorporated into the composite material of the present invention, however, in an alternative embodiment, the liner or topsheet 22 of the diaper 10 may also be treated with a surfactant to promote wettability of the liner, thereby promoting the wicking of moisture away from the surface of the user's skin and improved skin health conditions.

As noted above, in the alternative embodiment incorporating a surfactant, the fabric of the topsheet 36 may be surface treated with about 0.3 weight percent of a surfactant mixture which contains a mixture of AHCOVEL Base N-62 and GLUCOPON 220UP surfactant in a 3:1 ratio based on a total weight of the surfactant mixture. Other possible classes of surfactants include MASIL SF 19 and DC 193 Surfactant. The AHCOVEL Base N-62 is purchased from Uniqema (a division of ICI, and having offices in New Castle, Del.), and includes a blend of hydrogenated ethoxylated castor oil and sorbitan monooleate. The GLUCOPON 220UP is purchased from Cognis Corporation and includes an alkyl polyglycoside. MASIL SF 19 and DC 193 Surfactant are purchased from BASF (Gurnee, Ill.), and Dow Corning (Midland, Mich.), respectively. MASIL SF 19 and DC 193 Surfactant are examples of typical ethoxylated polyalkylsiloxanes. The surfactant may be applied by any conventional means, such as saturation, spraying, printing, roll transfer, slot coating, brush coating, internal melt addition or the like. The surfactant may be applied to the entire topsheet 22 or may be selectively applied to particular sections of the topsheet 22, such as the medial section along the longitudinal centerline of the diaper, to provide greater wettability of such sections.

The absorbent body 24 of the diaper 10, as representatively illustrated in FIGS. 1 and 2, may suitably comprise a matrix of hydrophilic fibers, such as a web of cellulosic fluff, mixed with particles of a high-absorbency material commonly known as superabsorbent material. In a particular embodiment, the absorbent body 24 comprises a matrix of cellulosic fluff, such as wood pulp fluff, and superabsorbent hydrogel-forming particles. The wood pulp fluff may be exchanged with synthetic, polymeric, meltblown fibers or with a combination of meltblown fibers and natural fibers. The superabsorbent particles may be substantially homogeneously mixed with the hydrophilic fibers or may be nonuniformly mixed. Alternatively, the absorbent body 24 may comprise a laminate of fibrous webs and superabsorbent material or other suitable means of maintaining a superabsorbent material in a localized area.

The absorbent body 24 may have any of a number of shapes and may consist of one or more regions or areas which may or may not all be in contact with one another. For example, the absorbent core may be rectangular, I-shaped, or T-shaped. When absorbent material is present in the intermediate section, it is generally preferred that the absorbent body 24 be narrower in the intermediate section than in the front or rear waist sections of the diaper 10. The absorbent body 24 may be provided by a single layer or, in the alternative, may be provided by multiple layers, all of which need not extend the entire length and width of the absorbent body 24. In a particular aspect of the invention, the article or system will have one or more regions which contain less absorbent material than other regions of the article. Although the most desired embodiment will have at least one region of the article in which the absorbent material is absent, it has been found that the presence of a reduced quantity of absorbent material provides some improvement, but not as much as complete removal or absence thereof, as some moisture will generally be absorbed and remain in the absorbent material that is present.

The size and the absorbent capacity of absorbent body 24 should be compatible with the size of the intended wearer and the liquid loading imparted by the intended use of the absorbent article. Further, the size and the absorbent capacity of the absorbent body 24 can be varied to accommodate wearers ranging from infants through adults. In addition, it has been found that with the present invention, the densities and/or basis weights of the absorbent body 24 can be varied.

The high-absorbency or superabsorbent material can be selected from natural, synthetic, and modified natural polymers and materials. The high-absorbency materials can be inorganic materials, such as silica gels, or organic compounds, such as crosslinked polymers.

Examples of synthetic, polymeric, high-absorbency materials include, but are not limited to, the alkali metal and ammonium salts of poly(acrylic acid) and poly(methacrylic acid), poly(acrylamides), poly(vinyl ethers), maleic anhydride copolymers with vinyl ethers and alpha-olefins, poly(vinyl pyrolidone), poly(vinyl morpholinone), poly(vinyl alcohol), and mixtures and copolymers thereof. Further polymers suitable for use in the absorbent core include natural and modified natural polymers, such as hydrolyzed acrylonitrile-grafted starch, acrylic acid grafted starch, methyl cellulose, carboxymethyl cellulose, hydroxypropyl cellulose, and the natural gums, such as alginates, xanthum gum, locust bean gum, and the like. Mixtures of natural and wholly or partially synthetic absorbent polymers can also be useful in the present invention.

The high absorbency material or superabsorbent may be in any of a wide variety of geometric forms. As a general rule, it is preferred that the high absorbency material be in the form of discrete particles. However, the high absorbency material may also be in the form of fibers, flakes, rods, spheres, needles, or the like. In general, the high absorbency material is present in the absorbent body in an amount of from about 5 to about 90 weight percent, desirably in an amount of at least about 30 weight percent, and even more desirably in an amount of at least about 50 weight percent based on a total weight of the absorbent body 24. For example, in a particularly embodiment, the absorbent body 24 may comprise a laminate which includes at least in part, and desirably at least about 50 weight percent and more desirably at least about 70 weight percent of high-absorbency material overwrapped by a fibrous web or other suitable means of maintaining the high-absorbency material in a localized area.

An example of high-absorbency material suitable for use in the present invention is HYSORB® P7050 polymer available from BASF, a business having offices in Portsmouth, Va. Other suitable superabsorbents may include, but are not limited to, DRYTECH® 2035 available from Dow Chemical Co. located in Midland, Mich., or FAVOR SXM 880 polymer obtained from Stockhausen, a business having offices in Greensboro, N.C.

As in conventional absorbent articles, due to the thinness of absorbent body 24 and the high absorbency or superabsorbent material within the absorbent body 24 of the present invention, the liquid uptake rates of the absorbent body 24, by itself, may be too low, or may not be adequately sustained over multiple insults of liquid into the absorbent body 24 (especially if one or more regions of an article or system are substantially free of absorbent material). To improve the overall liquid uptake and air exchange, a desired embodiment the present invention may further include the previously mentioned additional porous, liquid-permeable layer of surge management material 34, as representatively illustrated in FIGS. 1 and 2. The surge management layer 34 is typically less hydrophilic than the absorbent body 24, and has an operable level of density and basis weight to quickly collect and temporarily hold liquid surges, to transport the liquid from its initial entrance point and to substantially completely release the liquid to other parts of the absorbent body 24. This configuration can help prevent the liquid from pooling and collecting on the portion of the absorbent garment positioned against the wearer's skin, thereby reducing the feeling of wetness by the wearer. The structure of the surge management layer 34 also generally enhances the air exchange within the diaper 10. It is of note that this surge material is or may be in addition to the second, desirably superabsorbent-free, material or insert which may be in the one or more regions of the article containing less absorbent material than other regions.

Various woven and nonwoven fabrics can be used to construct the surge management layer 34. For example, the surge management layer 34 may be a layer composed of a meltblown or spunbonded web of synthetic fibers, such as polyolefin fibers. The surge management layer 34 may also, for example, be a bonded-carded-web or an airlaid web composed of natural and synthetic fibers. The bonded-carded-web may, for example, be a thermally bonded web which is bonded using low melt binder fibers, powder or adhesive. The webs can optionally include a mixture of different fibers. Although the layer of surge material may itself be made up of one or more layers of materials, for purposes of this disclosure the surge layer shall be referred to, for descriptive purposes only, as one layer. The surge management layer 34 may be composed of a substantially hydrophobic material, and the hydrophobic material may optionally be treated with a surfactant or otherwise processed to impart a desired level of wettability and hydrophilicity. In a particular embodiment, the surge management layer 34 includes a hydrophilic, nonwoven material having a basis weight of from about 30 to about 120 grams per square meter.

For example, in a particular embodiment, the surge management layer 34 may comprise a bonded-carded-web, nonwoven fabric which includes bicomponent fibers and which defines an overall basis weight of about 83 grams per square meter. The surge management layer 34 in such a configuration can be a homogeneous blend composed of about 60 weight percent polyethylene/polyester (PE/PET), sheath-core bicomponent fibers which have a fiber denier of about 2 and about 40 weight percent single component polyester fibers which have a fiber denier of about 3 and which have nominal fiber lengths of from about 3.8 to about 5.08 centimeters.

In the illustrated embodiments, the surge management layer 34 is preferably arranged in a direct, contacting liquid communication fashion with the absorbent body 24. The surge management layer 34 may be operably connected to the topsheet 22 with a conventional pattern of adhesive, such as a swirl adhesive pattern. In addition, the surge management layer 34 may be operably connected to the absorbent body 24 with a conventional pattern of adhesive. The amount of adhesive add-on should be sufficient to provide the desired levels of bonding, but should be low enough to avoid excessively restricting the movement of liquid from the topsheet 22, through the surge management layer 34 and into the absorbent body 24 or second material 25.

The absorbent body 24 is desirably positioned in liquid communication with surge management layer 34 to receive liquids released from the surge management layer, and to hold and store the liquid. In the shown embodiments, the surge management layer 34 comprises a separate layer which is positioned, at least in part, over another separate layer comprising the absorbent body 24 and/or the second material 25, thereby forming a dual-layer arrangement. The surge management layer 34 serves to quickly collect and temporarily hold discharged liquids, to transport such liquids from the point of initial contact and spread the liquid to other parts of the surge management layer 34, and then to substantially completely release such liquids into the layer or layers comprising the absorbent body 24.

The surge management layer 34 can be of any desired shape. Suitable shapes include for example, circular, rectangular, triangular, trapezoidal, oblong, dog-boned, hourglass-shaped, or oval. In certain embodiments, for example, the surge management layer can be generally rectangular-shaped. In the illustrated embodiments, the surge management layer 34 is coextensive with the absorbent body 24 and second or superabsorbent-free material 25, where the second material 25 is present. Alternatively, the surge management layer 34 may extend over only a part of the absorbent body 24. Where the surge management layer 34 extends only partially along the length of the absorbent body 24, the surge management layer 34 may be selectively positioned anywhere along the absorbent body 24. For example, the surge management layer 34 may function more efficiently when it is offset toward the front waist section 12 of the garment. The surge management layer 34 may also be approximately centered about the longitudinal center line of the absorbent body 24.

Additional materials suitable for the surge management layer 34 are set forth in U.S. Pat. No. 5,486,166 issued Jan. 23, 1996 in the name of Ellis et al. and entitled “Fibrous Nonwoven Web Surge Layer For Personal Care Absorbent Articles And The Like”; U.S. Pat. No. 5,490,846 issued Feb. 13, 1996 in the name of Ellis et al. and entitled “Improved Surge Management Fibrous Nonwoven Web For Personal Care Absorbent Articles And The Like”; and U.S. Pat. No. 5,364,382 issued Nov. 15, 1994 in the name of Latimer et al. and entitled “Absorbent Structure Having Improved Fluid Surge Management And Product Incorporating Same”, the disclosures of which are hereby incorporated by reference in their entirety.

As noted above, the majority of the disclosure herein is directed to an absorbent article and more specifically a diaper; however, the scope of the present invention is not to be limited thereby in any manner inconsistent with the claims. For example, although not illustrated, another embodiment of the present invention is directed to a composite system which defines a front waist section, a rear waist section, and an intermediate section which interconnects said front and rear waist sections, said intermediate section comprising a crotch region. The system including a superabsorbent-free material; and an absorbent, containing, at least in part a superabsorbent, wherein the absorbent is absent from one or more regions of the article and the superabsorbent-free material is positioned in one or more regions of the article adjacent the absorbent. It is of note that in at least one contemplated embodiment, the absence of absorbent in one or more regions of the article and the presence of the superabsorbent-free material in those regions promotes a reduction in time to move the insult from the superabsorbent-free material to the absorbent. The composite system is designed such that an insulting fluid is desirably moved or removed from the superabsorbent-free material to the absorbent material in less than about 2 minutes.

The present invention also contemplates a disposable absorbent article including a vapor permeable backsheet having a WVTR of at least about 1000 grams/m²/24 hr; a liquid permeable topsheet; an absorbent body located between the backsheet and the topsheet, the absorbent body located in one or more regions of the article; and a second material located between the backsheet and the topsheet, the second material being positioned in one or more regions of the article where the absorbent body is not present. The second material of the article allows fluid to be moved away from an insult area of the absorbent article to a region of the absorbent article where the absorbent is located so as to change the fluid profile in the diaper. Desirably the absorbent body is absent from the insult area of the absorbent article. Additionally, the disposable absorbent article may further include a vapor barrier positioned between the absorbent and the topsheet, the vapor barrier being a film, such that the vapor barrier assists in reducing the movement of moisture from the absorbent core into the area between the absorbent article and the user's skin, such that contact with the wearer's skin is minimized or reduced. Further still, it is desirable for the vapor permeable backsheet to be substantially liquid impermeable. The absorbent article may also be a personal care product, such as a diaper, training pant, absorbent underpant, adult incontinence product, feminine hygiene product, hygiene and absorbent product or the like.

The different embodiments of the present invention, as representatively illustrated in FIGS. 1-4 and described herein, advantageously provide improved absorbent articles which exhibit a change in the fluid profile of the articles and may also exhibit a reduction in skin-exudate contact and/or reduced saturation in the insult or target area. The reduced levels of liquid-skin contact promote drier, more comfortable skin and render the skin less susceptible to the viability of microorganisms. Thus, wearer's of absorbent articles made according to the present invention are believed to have reduced skin hydration which can lead to a reduction in the incidence of skin irritation and rash.

The following examples are presented to provide a more detailed understanding of the invention. The specific materials and parameters are exemplary and are not intended to specifically limit the scope of the invention.

### EXAMPLES

### Example 1

Disposable diapers having the same general construction as the HUGGIES® Ultratrim Step 3 diapers (described in more detail below, in connection with Examples 2-6) were tested. The diapers in Example 1 were not modified, except for the removal of the elastics, and were used as a control for the other Examples. The target was marked at 6 inches from the front of the absorbent pad. Table 1 illustrates the fluid distribution in the control after the second insult in accordance with the Bench Test described above.

[image]

Methodology for Examples 2-6

Disposable diapers having the same general construction as the HUGGIES® Ultratrim Step 3 diapers were tested. The diapers were substantially the same as the Ultratrim diapers except that a section of the absorbent was removed from the target area. In the design, the target was defined as being 6 inches from the front of the absorbent pad. One inch of absorbent was cut out from both sides of the target, the full width of the pad.

In the tested diapers, the backsheet included a microporous film/nonwoven laminate material comprising a spunbond nonwoven material laminated to a microporous film. The spunbond nonwoven comprised filaments of about 1.8 denier extruded from a polypropylene and defined a basis weight of about 20 grams per square meter. The film comprised a cast coextruded film having a calcium carbonate filled linear low polyethylene microporous core and ethylene vinyl acetate and Catalloy™ polypropylene (Catalloy™ 357P) blended skin layer having a basis weight of about 58 g rams per square meter prior to stretching. The film was preheated, stretched and annealed to form the micropores and then laminated to the spunbond nonwoven material. The resulting microporous film/nonwoven laminate based material had a basis weight of 45 grams per square meter and a water vapor transmission rate of about 4000 grams per square meter per 24 hours. Examples of such film/nonwoven laminate materials are described in more detail in U.S. Pat. No. 6,309,736, issued Oct. 20, 2001, in the name of McCormack et al. and en titled “Low Gauge Films And Film/Nonwoven Laminates”, the disclosure of which has been incorporated by reference in its entirety.

The absorbent core included an upper layer and a lower layer with the upper layer extending from the front edge of the absorbent core to a location about two thirds of the total length of the absorbent core. The absorbent core included from about 10 to about 11 grams of wood pulp fibers and from about 10 to about 11 grams of superabsorbent material and, accordingly, included about 50 weight percent wood pulp fibers and about 50 weight percent superabsorbent material. The lower layer had a basis weight of about 230 grams per square meter and the upper layer had a basis weight of about 560 grams per square meter to provide a total basis weight of about 790 grams per square meter in the front section of the core and a basis weight of about 230 grams per square meter in the back section of the core. The absorbent core further defined a width in the crotch section of about 6.35 centimeters.

The topsheet was comprised of a nonwoven, spunbond, polypropylene fabric composed of about 2.2-2.8 denier fibers formed into a web having a basis weight of about 17 grams per square meter and a density of about 0.11 gram per cubic centimeter. The topsheet was surface treated with an effective amount of a surfactant, such as about 0.3 weight percent of a surfactant commercially available from Uniqema under the trade designation AHCOVEL BASE N-62.

The surge management layer used in the Examples comprised a 2.5 osy homogenously blended bonded-carded-web, composed of 60 weight percent polyethylene/polypropylene (PE/PP), sheath-core bicomponent fibers which have a fiber denier of about 1.5 d (ESC215A 1.5 dpf HR6 (available from ES Fibervisions, having offices in Athens, Ga.) or ESC215 1.5 dpf HR6 (available from Chisso Corporation, having offices in Japan)) and 40 weight percent single component polyester fibers which have a fiber denier of about 3 d (Type 121 Merge 35351A or Type 235 Merge X52314 (both available from KoSa, having offices in Salisbury, N.C.)).

The diapers also included an elasticized leg band assembly along about two thirds of the length of each longitudinal side edge of the diaper. The assembly had six (6) strands of elastomeric material laminated to a breathable, nonwoven fabric layer. The elastic strands were composed of LYCRA® elastomer aligned along the longitudinal length of the diaper to elasticize and gather the diaper legbands. For testing purposes, the elastic leg band assemblies were completely cut out of the product to allow the product to lay flat.

In each of the Examples the fluid distribution was measured using a x-ray of the xy-plane of the insulted diaper. Tables 1-6, below, show the amount of fluid in 0.91 cm sections. These sections were measured in 0.91 cm longitudinal increments from the target area of the diaper. The total area of these sections will vary slightly, based on the CD width of the diaper in the area of measurement.

### Example 2

Disposable diapers having the same general construction as the Ultratrim Step 3 diapers were hand modified and tested in accordance with the methodology for Examples 2-6. In this example, the void created by the removal of the absorbent core from the area of the diaper was filled with a 2.5 osy surge (identified as “Insert A” in Table 2) having the same characteristics as the surge management layer discussed above. Specifically, Insert A is composed of 60 weight percent polyethylene/polypropylene (PE/PP) sheath-core bicomponent fibers which have a fiber denier of about 1.5 d (ESC215A 1.5 dpf HR6 or ESC215 1.5 dpf HR6) and about 40 weight percent single component polyester fibers which have a fiber denier of about 3 d (Type 121 Merge 35351A or Type 235 Merge X52314). The results are set forth in Table 2 below.

[image]

### Example 3

In this example, the void created by the removal of the absorbent core from the area of the diaper was filled with 175 gsm (5.1 osy) airlaid pulp (identified as “Insert B” in Table 3) composed of 90 weight percent NF 405 from Weyerhaeuser Company and 10 weight percent Type 255 Merge 34821A bicomponent from Hoechst-Celanese at a density of about 0.12 g/cc. The results are set forth in Table 3 below.

[image]

### Example 4

In this example, the void created by the removal of the absorbent core from the area of the diaper was filled with 5 layers of 30 gsm (0.88 osy) barrier tissue (identified as “Insert C” in Table 4) composed of 50 weight percent Millar Western BCTMP pulp and 50 weight percent Terrace Bay LL-19 pulp. The results are set forth in Table 4 below.

[image]

### Example 5

In this example, the void created by the removal of the absorbent core from the area of the diaper was filled with a 2.5 osy surge (identified as “Insert D” in Table 5) comprised of 60 weight percent polyethylene/polypropylene (PE/PP), sheath-core bicomponent fibers which have a fiber denier of about 0.9 d (Type 258 L1 (available from KoSa) or ESC201 HR6 (available from Chisso Corporation)) and about 40 weight percent single component polyester fibers which have a fiber denier of about 6 d (Type 295 L1 (available from KoSa)). The results are set forth in Table 5 below.

[image]

### Example 6

In this example, the void created by the removal of the absorbent core from the area of the diaper was filled with a 2.5 osy surge (identified as “Insert E” in Table 6) composed of 60 weight percent polyethylene/polyester (PE/PET), sheath-core bicomponent fibers of about 3 d (ESC233A (available from ES-Fibervisions) or ESC233 (available from Chisso Corporation)) and about 40 weight percent single component polyester fibers which have a fiber denier of about 6 d (Type 295 Merge 635020 (available from KoSa). The results are set forth in Table 6 below.

[image]

As illustrated in Tables 1-6, less fluid was held in the target area of each of the prototypes having at least some of the absorbent core removed.

The test results from Examples 1-6 indicate that diapers made according to the present invention generally have improved levels of fluid distribution and reduced saturation in the target.

### Example 7

Disposable diapers having the same general construction as the Ultratrim Step 3 diapers as described in connection with Example 2 were tested (as described below) as a control.

A second set of disposable diapers having the same general construction as the Ultratrim Step 3 diapers, as described above, were hand modified and tested. The diaper was substantially the same as the diaper of Example 2 except that a vapor barrier was inserted between the absorbent body and the topsheet. The barrier that was used was the same as that used for the outercover of the diaper. The outercover material (which was used as the vapor barrier) was positioned in the diaper with the film side towards the user's body. This material covered the absorbent and left an opening for the portion of the diaper containing less absorbent (e.g. the target area).

A product was prepared according to the procedure described with regard to Example 2 and marked in 3 inch sections for measurement. The product was placed into a cradle and insulted with 60 ml of saline in accordance with the Bench Test described above. After 1 minute, moisture readings were taken 3 inches in front of the target and 3 inches behind the target. The amount of moisture which passed from the absorbent body through the vapor barrier and the topsheet was measured and recorded. The amount of moisture was measured on the surface of the product after the first insult with a Wood Moisturemeter (AB® FMD-Plus moisture meter version 4.3) from Brookhuis Micro-Electronics. The moisture meter was connected to a hammer probe. No measuring pin was inserted into the probe as a surface measurement was desired, and if the measuring pins were used, they would penetrate the product. All measurements are in percent moisture and are shown in Table 7 below.

TABLE 7

| | | |
|---|---|---|
| Distance From Target | −3″ | +3″ |

CONTROL

| | | |
|---|---|---|
| Location, insult | x_{1,1} | x_{3,1} |
| Control Run 1 | 24.2 | 71.1 |
| Control Run 2 | 50.4 | 70.4 |
| Control Run 3 | 33 | 72.1 |
| Average | 36 | 71.2 |
| St. Dev. | 13 | 0.9 |

With Barrier

| | | |
|---|---|---|
| Location, insult | x_{1,1} | x_{3,1} |
| Diaper + Barrier (Run 1) | 10.6 | 10.6 |
| Diaper + Barrier (Run 2) | 10.5 | 12.4 |
| Diaper + Barrier (Run 3) | 7.3 | 13.1 |
| Average | 9.5 | 12.0 |
| St. Dev. | 1.9 | 1.3 |

While various patents and other reference materials have been incorporated herein by reference, to the extent there is any inconsistency between incorporated material and that of the written specification, the written specification shall control. In addition, while the invention has been described in detail with respect to specific embodiments thereto, it will be readily apparent to those skilled in the art that various alterations, modifications and other changes can be made without departing from the spirit and scope of the present invention. It is therefore intended that the claims cover or encompass all such alterations, modifications and/or changes.

We claim:

1. An absorbent article which defines a front waist section, a rear waist section, and an intermediate section which interconnects said front and rear waist sections, said absorbent article comprising:
an absorbent material containing, at least in part, a superabsorbent; and
a second material;
wherein one or more regions of the article contains less absorbent material than other regions of the article, and wherein the second material is positioned in at least one of the one or more regions of article which contain less absorbent material.

2. The absorbent article of claim 1, wherein the reduction of absorbent material and the positioning of the second material in one or more regions of the article promote flow of an insulting liquid from the one or more regions of the article containing less absorbent material to other regions of the article so as to change the fluid profile in the article.

3. The absorbent article of claim 1, wherein the absorbent material is absent from one or more regions of the article.

4. The absorbent article of claim 1, wherein the intermediate section comprises, at least in part, a crotch region and wherein the absorbent material is absent from the crotch region of the article.

5. The absorbent article of claim 1, wherein the second material is selected from one or more of the group consisting of surge, tissue, or airlaid materials.

6. The absorbent article of claim 1 further comprising one or more of the following:
a additional surge material, said additional surge material being positioned between the topsheet and the second material or the absorbent material; and
a vapor barrier, said vapor barrier positioned between the absorbent and the topsheet; wherein said vapor barrier reduces the amount of moisture exposed to the skin of a wearer.

7. The absorbent article of claim 1, wherein the backsheet may be comprised of a highly breathable laminate.

8. The absorbent article of claim 1, wherein the highly breathable laminate is a film/nonwoven laminate.

9. The absorbent article of claim 8, wherein the nonwoven is a spunbond.

10. The absorbent article of claim 6, wherein the vapor barrier is a film.

11. The absorbent article of claim 1, wherein the backsheet has a Water Vapor Transmission Rate of at least 2,500 g/m²/24 hr.

12. A composite system which defines a front waist section, a rear waist section, and an intermediate section which interconnects said front and rear waist sections, said system comprising:
a superabsorbent-free material; and
an absorbent, containing, at least in part a superabsorbent,
wherein the absorbent is absent from one or more regions of the article; and
wherein the superabsorbent-free material is positioned in one or more regions of the article adjacent the absorbent.

13. The composite system of claim 12, wherein the absence of absorbent in one or more regions of the article and the presence of the superabsorbent-free material in those regions promotes a reduction in time to move the insult from the superabsorbent-free material to the absorbent.

14. The composite system of claim 12, wherein the insult is removed from the superabsorbent-free material to the absorbent material in less than about 2 minutes.

15. The composite system of claim 12, wherein the superabsorbent-free material comprises surge, tissue or airlaid materials.

16. A disposable absorbent article which comprises:
a vapor permeable backsheet which defines a Water Vapor Transmission Rate of at least about 1000 grams per square meter per 24 hours calculated according to a Water Vapor Transmission Test as set forth herein;
a liquid permeable topsheet;
an absorbent body located between said backsheet and said topsheet, said absorbent body located in one or more regions of the article; and
a second material located between said backsheet and said topsheet, said second material being positioned in one or more regions of the article where the absorbent body is not present.

17. The absorbent article of claim 16, wherein second material allows fluid to be moved away from an insult area of the absorbent article to a region of the absorbent article where the absorbent is located so as to change the fluid profile in the diaper.

18. The absorbent article of claim 16, wherein the absorbent body is absent from the insult area of the absorbent article.

19. The disposable absorbent article of claim 16 further comprising a vapor barrier positioned between the absorbent and the topsheet.

20. The absorbent article of claim 19, wherein the vapor barrier is a film, and wherein the vapor barrier reduces the evaporation from the absorbent core into the absorbent article.

21. The absorbent article of claim 16, wherein the second material is selected from the group consisting of surge, tissue or airlaid materials.

22. The absorbent article of claim 16, wherein said vapor permeable backsheet is substantially liquid impermeable.

23. The absorbent article product of claim 16 is a personal care product.

24. The personal care product of claim 23, wherein the personal care product is selected from a diaper, training pant, absorbent underpant, adult incontinence product, feminine hygiene product, and hygiene products.
